(19) United States
(12) Patent Application Publication
(10) Pub. No.: US 2002/0013592 A1
(43) Pub. Date: Jan. 31, 2002

(54) Device for closing an anastomosis between the aorta and the pulmonary artery

(75) Inventor: Peter Osypka, Grenzach-Wyhlen (DE)

Correspondence Address: VOLPE AND KOENIG, P.C., SUITE 400, ONE PENN CENTER, 1617 JOHN F. KENNEDY BOULEVARD, PHILADELPHIA, PA, 19103, US

(73) Assignee: Dr. Osypka GmbH, Rheinfelden (DE)

(21) Appl. No.: 09/908,111

(22) Filed: Jul. 18, 2001

(30) Foreign Application Priority Data
Jul. 20, 2000 (DE) 100 35 230.8

(51) Int. Cl. A61B 17/08

(52) U.S. Cl. 606/158

(57) ABSTRACT

A device (1) for closing a canal-shaped anastomosis (2), between the aorta (3) and the pulmonary artery (4), which remained open after the birth of a person (6). It is formed by a closing element (8) that can be inserted using a catheter (7) and that has two seals (10) and (11) spaced apart from each other and connected via an elastic tension element (9). The two seals have a certain elasticity and in the usage position, cover or fill up the two mouths or ends of the anastomosis (2), located between the aorta (3) and the pulmonary artery (4). The seals (10) and (11) can be folded, so that they on the one hand fit into the catheter (7) and on the other hand, can be introduced through the anastomosis (2) and brought to the respective application position. Using an eyelet (16) that is arranged in the area of the entrance into an inner longitudinal cavity (12) of the device and a tension thread (17) that passes through it, the seating of the closing element can be corrected especially through altematingly activating the thread (17) and an obturator or tool.

### BACKGROUND

The invention involves a device for closing an anastomosis between the aorta and the pulmonary artery which remained open after birth, with a sealing element that can be inserted into this anastomosis using a catheter, whereby the sealing element has two seals that can be set off at a distance from each other and are connected elastically via a tension element and, in the usage position, cover or fill up the two mouths of the anastomosis located between the aorta and the pulmonary artery.

It is known that an anastomosis between the aorta and the pulmonary artery which is at first open after birth normally closes by itself. In some cases, this anastomosis remains open, however, and must then be artificially closed, which until now has been done surgically.

From the U.S. Pat. No. 5,192,301, a device of the above-noted type is known. The sealing element can be brought into its position with a push wire. There is the danger in this, that the best possible positioning of the sealing element is not made.

When using this system on the pulmonary artery, parts can be produced that possibly project out into the artery, on which undesired blood dam age can occur and possibly a clot can form.

### SUMMARY

Therefore, the object of the invention is to create a device of the above-noted type, with which an anastomosis between the aorta and pulmonary artery, which remains open after birth, can be sealed shut, whereby after the introduction of the sealing element, the best possible placement can be sought and achieved.

The object of the invention is achieved in that the device is essentially formed using a sealing element that can be inserted using a catheter, with two seals set at a distance from each other, which is characterized in that one seal and the tension element have an inner longitudinal cavity that extends into the vicinity of the other seal, for the entrance of an obturator or equivalent pushing and/or turning tool, and this cavity is reinforced and/or stiffened on its end, and that in the area of the entrance into the inner longitudinal cavity of the tension element, a reinforcement is arranged on which an eyelet that extends outwardly over the seal that is immediately adjacent, and a tension thread that is suspended on it, are arranged.

In this manner, a corresponding sealing element can be brought into the opening using a catheter without a surgical opening of the rib cage, where the opening is closed on both sides independently of its length. The elastic tension element arranged between the two seals thus allows an adaptation to the differences in length of the corresponding opening. At the same time, both seals are pressed through this elastic tension element onto their “seat” and produce a good seal. Thus, a good adaptation can be achieved both to the length as well as also to the diameter of the opening, and a residual flow can be prevented between the aorta and the pulmonary artery.

The reinforced inner longitudinal cavity thus allows the use of an obturator or equivalent tool during insertion. The tension thread arranged on the eyelet on the proximal seal or “sheath” can be considered as a “safety line”. This thread can be removed by pulling on one end once it has been checked that the seals of the sealing element are correctly placed after the introduction of the sealing element. Otherwise, the entire seal can be removed by pulling on both ends of this thread that is guided through the eyelet and the catheter can be removed again. Also, by altematingly activating the tension thread and the obturator or tool, the best possible positioning can be sought and achieved. The sealing element can thus be introduced from one side into the opening that is to be closed and thus positioned so that the one seal reaches the end that is further away in the insertion direction and/or the mouth of this opening, and closes it. The second seal then covers the entrance opening and the insertion tool can be pulled back again.

It is advantageous in the process if the reinforcement in the area of the entrance into the inner longitudinal cavity of the tension element is a helix preferably made out of metal wire. Thus, it provides a good mount especially for the eyelet during the application of tension forces.

It is also advantageous in the process if the seals and the tension elements that connect them are made out of an elastic synthetic material, especially out of silicone material. It has been proven over many years that silicone is allergy-free and physically compatible, for example, even for implantable heart pacemaker electrodes. At the same time, it is rubber-elastic and can therefore automatically adapt the seals themselves and the tension element(s) based on anatomical conditions.

The two seals and the tension element that connects them elastically can be connected as a single piece. The introduction and placement of this seal element is also correspondingly simple.

The two seals of the sealing element can be constructed in the shape of a plate or a sheath and can be somewhat flat or at least sunk in a concave manner in certain areas on their outer surface. In this way, as opposed to seals that are arched to the outside, a constriction of the cross-section of the aorta and the pulmonary artery is prevented.

For a secure introduction of the sealing element, it is preferable if the inner longitudinal cavity of the tension element is reinforced on the inside, in particular by a helix, for example, made of metal wire, and that the reinforcement forms or surrounds on its end a profiling that is hollow on the inside, for example, a slot into which the flat end of the obturator or tool fits. Thus, the user can shove the sealing element forward with the help of the obturator or tool and thus, possibly also turn it somewhat until the seals have reached their prescribed position on both mouths of the opening that lie at a distance from each other, and close them securely.

The sheath-like seals can be folded against the elasticity of their material and can be inserted into a tube-like supply catheter—which can be a part of the device, the inner cross-section of which corresponds to the outer dimension of the seals that are folded together. Thus, the entire sealing element can be brought inside the catheter in the area of the opening with both seals at first in a folded arrangement and there, they are shoved using an obturator or tool out of the supply catheter into the opening.

The sheath-like seals can have a round or somewhat square or oval circumferential contour and their surface can have, in particular, reinforced ribs and/or struts. Thus, adaptations to different anatomical conditions are possible in the area of the opening that is to be closed.

An additional embodiment of the invention for increasing the certainty of the seal of the opening that is to be closed can be provided in that the tension element between the two end side seals carries at least one additional elastic seal, which can be adapted elastically at least in its edge area. This additional seal can thus rest on the inner wall between the two end seals in the progression of the somewhat canal-shaped opening that is to be closed, and thus further stop an undesired blood flow through this opening.

In this manner, two additional seals that are set apart from each other at a distance can be arranged on the tension element between the two end seals, the outer diameter of which is, in particular, smaller than that of the end seals, so that they fit into the inside of the canal-shaped opening.

The additional seal(s) along the progression of the tension element can also be somewhat sheath-like and have a sheath-like or conical shape that can be folded against a restoring force of the material. Thus, they can also be housed inside the supply catheter.

The selection of the sealing shape and size can possibly be determined prior to the intervention, for example through ultrasound. Also, the sheath-like seals can be cut to size after determining the size of the opening, or “ductus”, that is to be closed, which can be performed, especially when the manufacture is made from silicone, with simple scissors on the operating table.

On the whole, a seal that can be introduced in a simple way results, whose two seals themselves provide for a secure and impermeable mounting even when there are large anatomical anomalies.

### BRIEF DESCRIPTION OF THE DRAWINGS

In the following, the preferred embodiments are described in greater detail using the drawing. Shown in partially schematic diagrams are:

FIG. 1 is a view of person and his heart, in which an anastomosis between the aorta and the pulmonary artery is shown, which has remained open after birth, and which is closed subsequently with a sealing element that can be introduced with a catheter,

FIG. 2 is an enlarged scale, cross-section through the heart and through parts of the aorta and the pulmonary artery with an anastomosis of these two blood vessels that remains open and that can be subsequently closed using an sealing element according to the invention,

FIG. 3 is an enlarged scale view of the sealing element in the usage position, whereby the sealing element has two elastically connected seals for the mouths or ends of the anastomosis between the aorta and the pulmonary artery, which are set at a distance from each other and connected elastically using tension elements,

FIG. 4 is an enlarged scale view of the detail marked with the circle A in FIG. 3, and

FIGS. 5 to 18 show plan views and side views of seals having different outer contours and for to the sealing element.

### DETAILED DESCRIPTION OF THE PREFERRED EMBODIMENTS

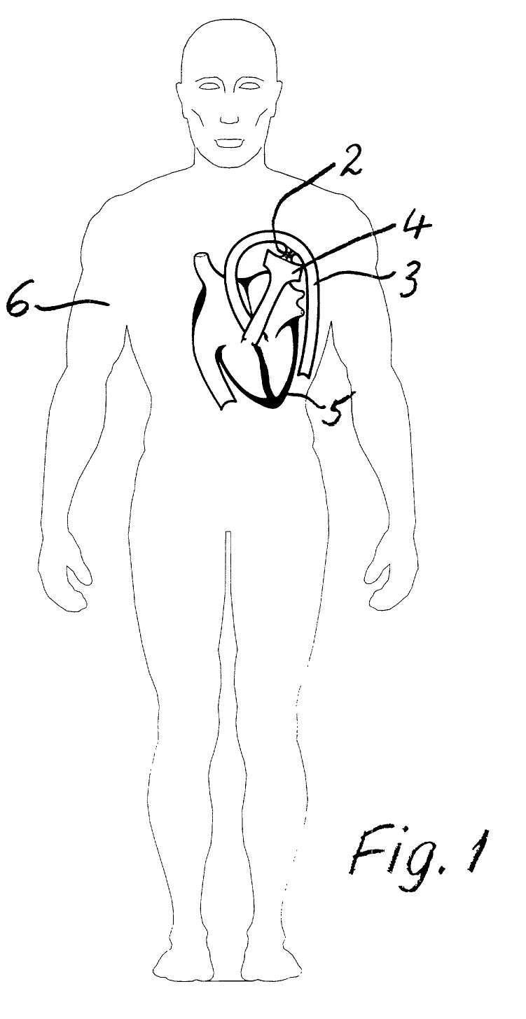

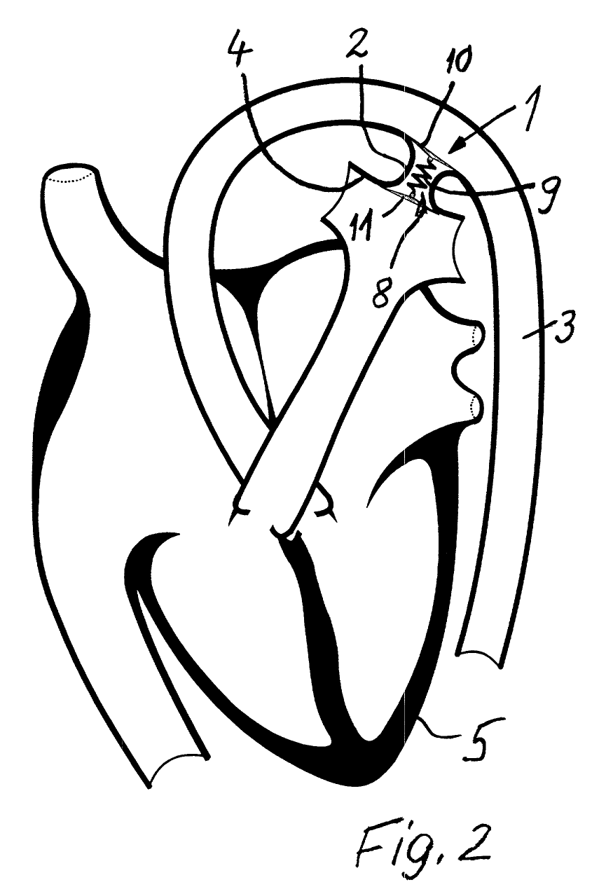

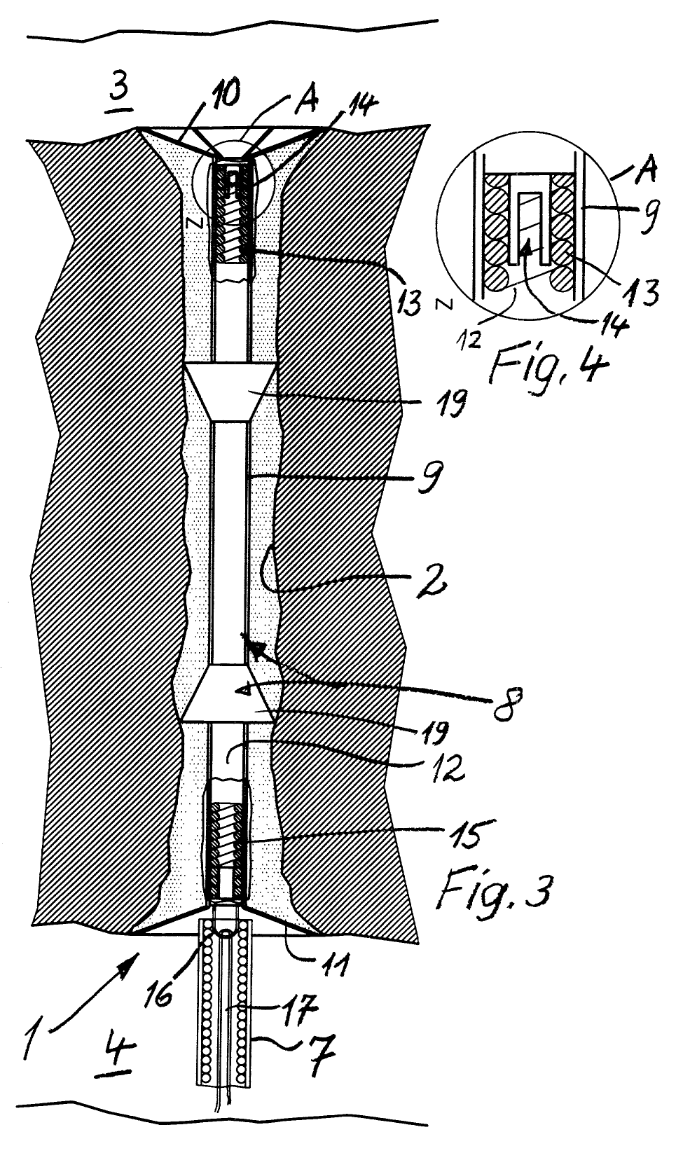

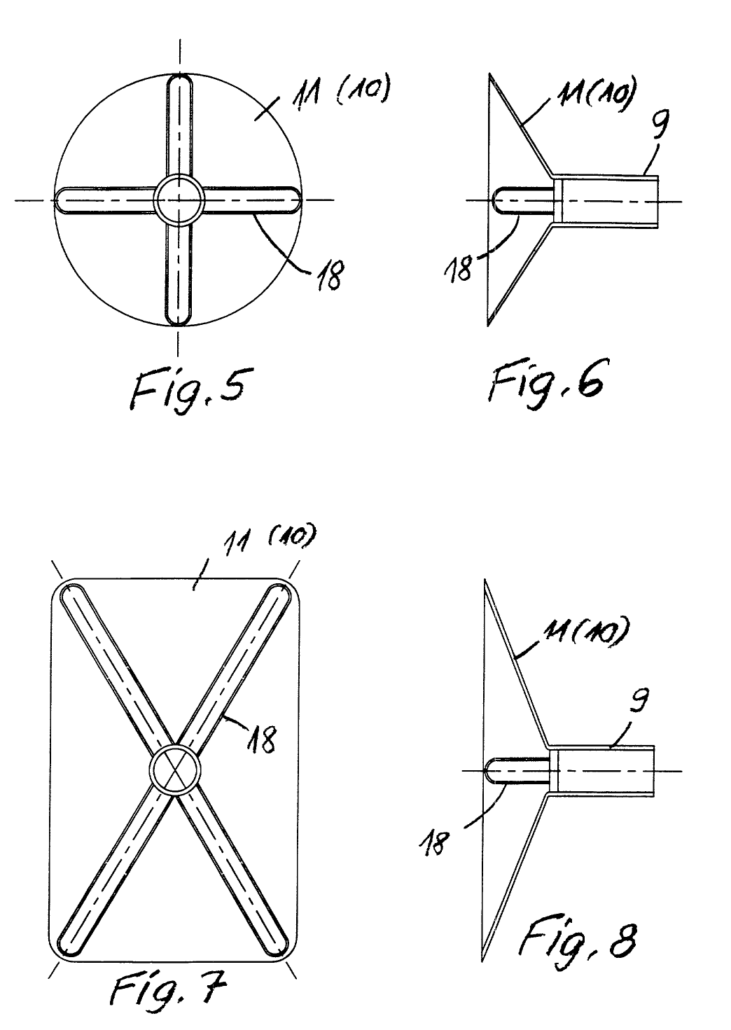

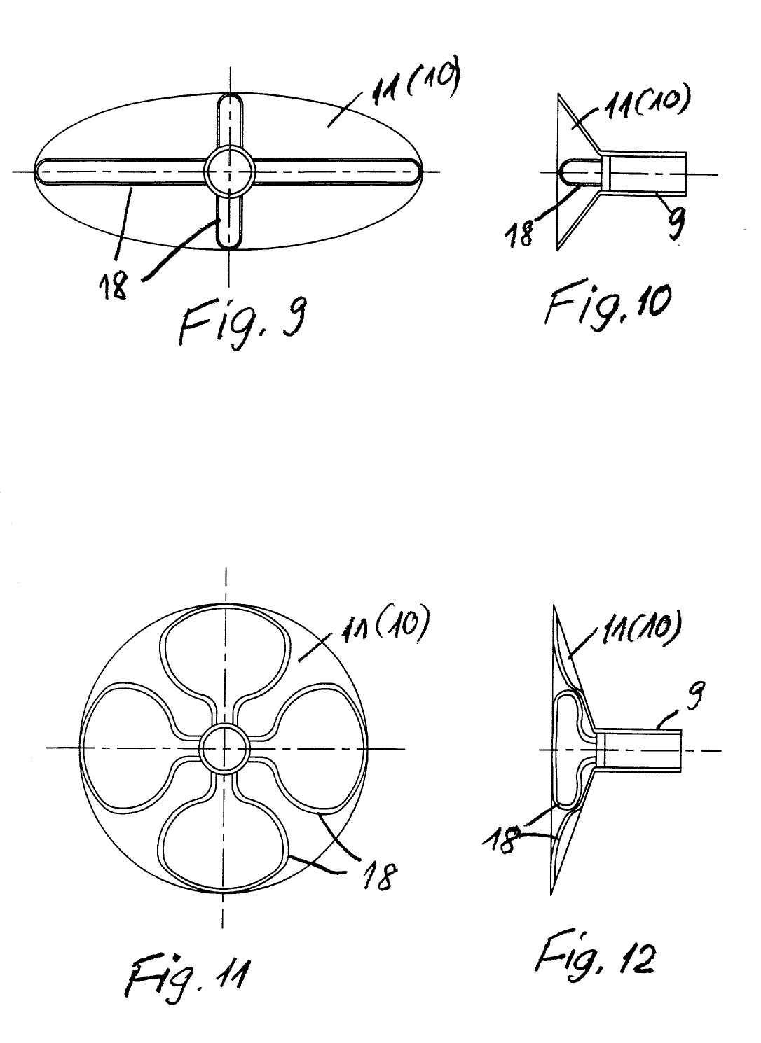

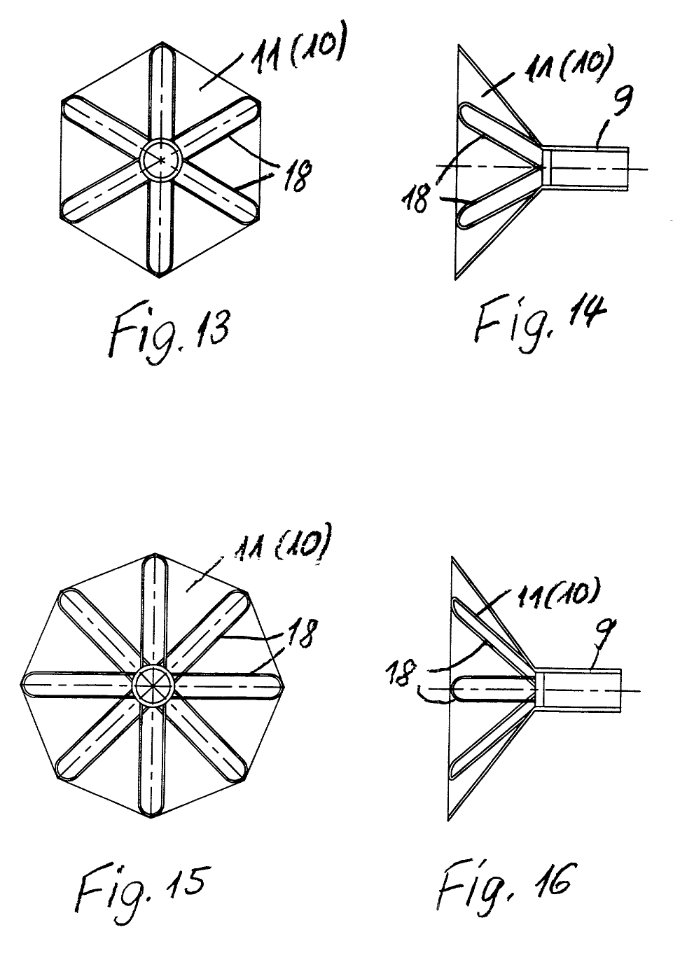

A device that is indicated as a whole by 1 and can be recognized especially well in FIG. 3 functions for the closing of an anastomosis 2 between the aorta 3 and the pulmonary artery 4, which has remained open after birth, whereby this anastomosis 2 can be recognized as depicted schematically in FIG. 1 and 2 on the heart 5 of a person 6. The device 1 is formed essentially through a sealing element 8 that can be introduced using a catheter 7, so that a surgical operation to close the anastomosis 2 can be avoided. From the catheter 7 mentioned, which only has an end indicated in FIG. 3, with this end being designed so that it can accommodate the entire sealing element 8, in order to be able to be mounted at the insertion position through blood vessels.

FIG. 2, and especially FIG. 3, show that the closing element 8 has two seals 10 and 11 which though set apart at a distance, are connected via an elastic tension element 9, and which in the usage position cover or fill up the two mouths of the anastomosis located between the aorta 3 and the pulmonary artery 4. FIG. 3 shows that these mouths of the anastomosis 2 are expanded somewhat opposite the remaining progression of this canal-shaped anastomosis 2, but in spite of that, are covered by the seals 10 and 11 so that they stop liquid from coming through.

Whereas FIG. 2 shows schematically that the two seals 10 and 11 can be connected through a spring as a tension element 9, it is provided according to FIG. 3 that the seals 10 and 11 and the tension element 9 connecting them are made of an elastic synthetic material, preferably a silicone material, which has proven to be inert and allergy-free when used within the human body.

The two seals 10 and 11 and the tension element 9 that elastically connects them are shown as a single piece in the embodiment of FIG. 2. The two seals 10 and 11, which each can have approximately the same shape and the same design and of which different examples are shown in FIGS. 5 to 18 in terms of the shape and design, are each constructed in a sheath-like manner, i.e. they can folded together to a considerably smaller cross-section than the shapes shown and the arrangement in FIG. 3, so that they fit into the catheter 7. In the usage position, they are flat, or, as shown in the illustrated embodiments, sunken in on their outer surface, so that on both blood vessels 3 and 4, no projection results through these seals 10 and 11.

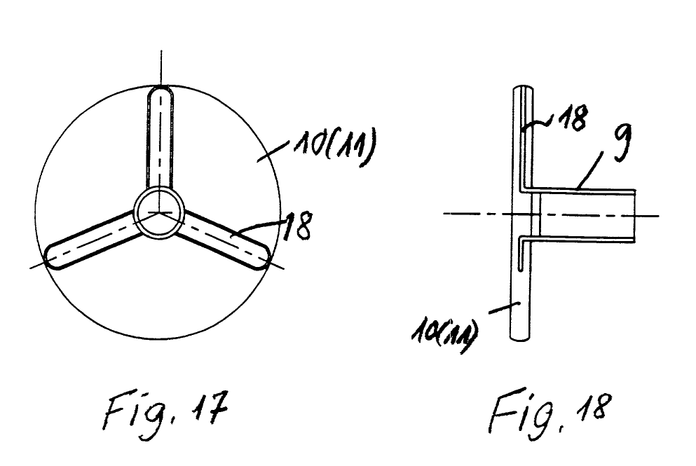

In FIG. 3 it is recognized that the one seal 11, namely the proximal seal 11 and the tension element 9, has an inner longitudinal cavity 12, extending up to the vicinity of the other seal 10, which is for the entrance of an obturator or equivalent pushing and/or turning tool, and which is reinforced and stiffened at least on one end in the area of the detail A (see FIG. 4). Thus, through the catheter 7, a corresponding pushing tool that is not shown in greater detail is inserted through the sealing element 8 Lip to the vicinity of the seal 10, in order to push the sealing element out of the catheter 7 into the anastomosis 2. The seal 10 is thus distinguished from the seal 11 in that it has no such entrance opening for a tool of this sort.

The inner longitudinal cavity 12 of the tension element 9 is thus reinforced on the inside through a helix 13, for example, made out of a metal wire, and this reinforcement forms on its end near the seal 10 a profiling that is hollow on the inside, which contains in addition a slit 14, as shown in FIG. 4, in which a correspondingly flat end of the obturator or tool fits, so that rotational movements are also possible so that the seal 10 or the entire sealing element 8 can be adapted to certain anatomical conditions.

The sheath-like seals 10 and 11 can be folded against the elasticity of their material in a manner not shown, and they can be inserted into the tube-like supply catheter 7 before the sealing element 8 is brought to its insertion position, The inner cross-section of this supply catheter 7 thus corresponds to the outer dimensions of the folded seals 10 and 11.

In FIG. 3, it will also be recognized in the area of the entrance into the longitudinal cavity 12 of the tension element 9, i.e. near to the seal 11, a reinforcement in the form of a second helix 15, made of metal wire, for example. In this way, the tension element 9 is stabilized in particular near to both seals 10 and 11. Furthermore, it is possible by this that on the helix 15, an eyelet 16 can be provided over the adjacent seal 11 that projects outwardly, on which a tension thread 17 is threaded and suspended. Using this tension thread 17, by a pulling force on its two ends, the entire sealing element 8 can be pulled out of the anastomosis 2 again, if, for example, a test with ultrasound shows that the placement does not agree or the sealing ability was not achieved with the necessary certainty or the seals 10 and 11 do not fit exactly.

If a sealing element 8 is used that seals well and fits well, the tension thread 17 can be removed by pulling one end out of the eyelet 16.

FIGS. 5 to 18, already mentioned several times, show that the seals 10 and 11 that are sheath-like, are opposingly concave-shaped (FIG. 5 to 16) like an umbrella, or are flat (FIG. 17 and 18), can have the most diverse circumferential contours, for example round, square, multi-sided, or oval, and that their surfaces have reinforcement ribs or struts 18, respectively, possibly of different number. It will be recognized, extending outwardly from the center, either three (FIG. 17) or four (FIG. 5 to 12) or even more (FIG. 13 to 6) of these reinforcement ribs or struts 18 can be provided. These ribs can, for their part, be shaped differently, as is indicated especially in FIG. 11 in comparison to the other Figures. Thus, the rigidity and adaptability of the seals 10 and 11 can be prespecified or selected according to the application case and anatomical conditions. Furthermore, the circumferential contours can also be cut by the surgeon, in particular, in the edge areas located between the reinforcement ribs or struts 18.

In FIG. 3, furthermore, it will be recognized that the tension element 9 carries or has between the two end seals 10 and 11 at least one elastic seal, and in the second embodiment in FIG. 3, elastic seals 19 that are set at a distance from each other, which can be elastically adapted at least in their edge areas, and can thus automatically adapt and rest on the inner cross section of the canal-shaped anastomosis 2. In this case, their outer diameter in the disclosed embodiment is smaller than that of the end seals 10 and 11, in order to take into account the cross-section that is usually smaller than the ends or mouths of this anastomosis 2. It is especially well shown by FIG. 1, that in this way, liquid is to the greatest extent possible prevented from getting through from one of the blood vessels to the other one.

The additional seals 19 are conical according to FIG. 3 and can in this manner also be somewhat sheath-like and folded against a restoring force of the material, in order to be able to adapt even better to different inner contours within the anastomosis 2.

After the size and the shape of the anastomosis 2 or its ductus has been determined by ultrasound, a fitting closing element 8 can thus be selected and inserted using the catheter 7. If necessary, the circumferential contour of the seals 10 and 11 can be cut to size in advance on the operating table. During the introduction phase, the device 1 is housed folded together in the protection or insertion catheter 7 and is pushed to the desired position and squeezed out there with an obturator or pushing tool. The flattened out end of this tool fits into the distal receptor slot 14 on the end of the inner longitudinal cavity 12 of the tension element 9, so that the distal seal 10 can also be turned if necessary into the “correct” position. The already mentioned eyelet 16 with thread 17, which practically represents a “safety line”, is advantageous. This thread 17 is removed by pulling on one of its ends only if, after the placement of the sealing element 8, it had been checked to see that it is correctly seated. The sheath-like seals 10 and 11 on both ends of the sealing element 8 themselves provide for a secure and impermeable attachment even when there are large anatomical anomalies.

The device 1 functions for closing a canal-shaped amastomosis 2, between the aorta 3 and the pulmonary artery 4, which remained open after the birth of a person 6. It is formed essentially by a closing element 8 that can be inserted using a catheter 7, which has two seals 10 and 11 set apart from each other at a distance and connected via an elastic tension element 9, which for their part have a certain elasticity and in the usage position cover or fill up the two mouths or ends of the anastomosis 2, located between the aorta 3 and the pulmonary artery 4, whereby it is advantageous if these seals 10 and 11 can be folded, so that they on the one hand fit into the catheter 7 and on the other hand, can be introduced through the anastomosis 2 and brought to the respective application position. Using an eyelet 16 that is arranged in the area of the entrance into the inner longitudinal cavity 12 and a tension thread 17 that passes through it, the seat of the sealing element can be corrected especially through altematingly activating the thread 17 and the obturator or tool.

What is claimed is:

1. Device (1) for closing an anastomosis (2) having two mouths, between the aorta (3) and the pulmonary artery (4), which remained open after birth, comprising a closing element that can be inserted using a catheter (7), the closing element (8) includes two seals (10, 11) set apart from each other at a distance and connected elastically via a tension element (9), which in a usage position, is adapted cover or fill up the two mouths of the anastomosis (2), located between the aorta (3) and the pulmonary artery (4), one of the seals (11) and the tension element (9) have an inner longitudinal cavity (12) that extends in proximity to the other seal (10), to define an entrance for an obturator or equivalent pushing and/or turning tool, where the cavity is reinforced and/or stiffened on one end, and in an area of the entrance into the inner longitudinal cavity (12) of the tension element (9), a reinforcement is arranged having an eyelet (16) that extends outwardly from the seal (11) that is immediately adjacent, and a tension thread (17) is suspended on the eyelet.

2. Device according to claim 1, wherein the reinforcement in the area of the entrance into the inner longitudinal cavity (12) of the tension element (9) is a helix (15), preferably made out of metal wire.

3. Device according to claim 1, wherein the seals (10) and (11) and the tension element (9) connecting them are made of an elastic synthetic material, preferably a silicone material.

4. Device according to one of the claim 1, wherein the two seals (10,11) and the tension element (9) connecting them are connected as a single piece.

5. Device according to claim 1, wherein the two seals (10,11) of the closing element are formed in a plate-like or a sheath-like shape and are somewhat flat or at least sunk in a concave manner in certain areas on an outer surface thereof.

6. Device according to claim 1, wherein the inner longitudinal cavity (12) of the tension element (9) is reinforced on an inside thereof, and the reinforcement forms or surrounds on an end thereof a profiling or a slot that is hollow on the inside, into which a flat end of the obturator or tool is adapted to fit.

7. Device according to claim 6, wherein the inner longitudinal cavity (12) of the tension element (9) is reinforced on the inside by a helix, made of metal wire.

8. Device according to claim 1, wherein the sheath-like seals (10, 11) can be folded against an elasticity of their material and are adapted to be inserted into a tube-like supply catheter (7), an inner cross-section of which corresponds to an outer dimension of the folded seals (10, 11).

9. Device according to claim 1, wherein the sheath-like seals (10, 11) have one of a round, a generally square, polygonal, or oval circumferential contour and have a surface with reinforced ribs and/or struts (16).

10. Device according to claim 1 wherein the tension element (9) carries at least one additional elastic seal (19), between the two end seals (10, 11), which is elastically adjustable at least in an edge area.

11. Device according to claim 1, wherein two additional seals (19) that are spaced apart from each are arranged on the tension element (9) between the two end seals (10, 11), the two additional seals have an outer diameter which is smaller than an outer diameter of the end seals (10, 11).

12. Device according to claim 10, wherein the additional seal (19) has a generally sheath-like or conical shape and is adapted to be folded against a restoring force of the material.
